(19) United States
(12) Patent Application Publication
(10) Pub. No.: US 2018/0199839 A1
(43) Pub. Date: Jul. 19, 2018

(54) INTRATHORACIC PRESSURE CALCULATION DEVICE AND INTRATHORACIC PRESSURE CALCULATION METHOD

(71) Applicant: DENSO CORPORATION, Kariya-city, Aichi-pref. (JP)

(72) Inventors: Rie OSAKI, Kariya-city (JP); Shinya KUROSAWA, Kariya-city (JP)

(21) Appl. No.: 15/743,579

(22) PCT Filed: Jul. 12, 2016
(86) PCT No.: PCT/JP2016/070547

§ 371 Date: Jan. 10, 2018

(30) Foreign Application Priority Data
Aug. 6, 2015 (JP) 2015-155905

(51) Int. Cl. A61B 5/03; A61B 5/087; A61B 5/021

(52) U.S. Cl. CPC A61B 5/03; A61B 5/0205; A61B 5/02116; A61B 5/087

(57) ABSTRACT

An intraoral pressure signal is associated with a pulse wave signal along a time axis. As a calibration coefficient, a ratio of a variation in an amount of change in an amplitude of the pulse wave signal from a preset second reference value to a variation in an amount of change in the intraoral pressure represented by the intraoral pressure signal from a preset first reference value is calculated based on the acquired pulse wave signal and the acquired intraoral pressure signal. An absolute value of an intrathoracic pressure of the subject is calculated by multiplying an estimated intrathoracic pressure, which is a relative value of the intrathoracic pressure estimated based on the pulse wave signal, by the calibration coefficient.

### CROSS REFERENCE TO RELATED APPLICATION

The present application is based on Japanese Patent Application No. 2015-155905 filed on Aug. 6, 2015, the disclosure of which is incorporated herein by reference.

### TECHNICAL FIELD

The present disclosure relates to a technique for calculating an intrathoracic pressure.

### BACKGROUND ART

Conventionally, a known intrathoracic pressure calculation device is provided with a pulse wave acquisition unit that acquires a pulse wave signal representing a pulse wave of a subject and an estimation unit that estimates an intrathoracic pressure of the subject based on the pulse wave signal acquired with the pulse wave acquisition unit (refer to Patent Literature 1).

The estimation unit of the intrathoracic pressure calculation device disclosed in Patent Literature 1 creates a first envelope that connects peaks of the pulse wave of one beat represented by the pulse wave signal, and creates a second envelope that connects peaks of the first envelope. The estimation unit estimates a difference between the first envelope and the second envelope as an intrathoracic pressure signal representing an intrathoracic pressure of the subject.

### PRIOR ART LITERATURE

### Patent Literature

PATENT LITERATURE 1: JP-A-2002-355227

Incidentally, the intrathoracic pressure signal estimated with the intrathoracic pressure calculation device disclosed in Patent Literature 1 represents a transition of a pressure due to a relative change, and indicates a relative value of the intrathoracic pressure. In order to convert the relative value of the intrathoracic pressure to an absolute value, there is a need to perform calibration.

The calibration is implemented by multiplying the intrathoracic pressure signal by a calibration coefficient. The calibration coefficient is calculated in advance on the basis of a correspondence relationship between an intraoral pressure of the subject measured in advance and the intrathoracic pressure signal, on assumption that the intraoral pressure of the subject is equal to the intrathoracic pressure of the subject.

However, in case where a resistance between an oral cavity and a thoracic cavity is large due to diseases such as airway obstruction, a loss increases, and the intraoral pressure and the intrathoracic pressure do not become equal to each other. As described above, in case where the calibration is executed with the use of the calibration coefficient calculated under a condition that the intraoral pressure and the intrathoracic pressure do not become equal to each other, it is concerned that an accuracy of the intrathoracic pressure corrected by the calibration is low. In other words, an improvement in calculation accuracy in a technique for obtaining an absolute value of intrathoracic pressure is considered to be one issue.

### SUMMARY OF INVENTION

It is an object of the present disclosure to provide a technique for improving a calculation accuracy of an intrathoracic pressure.

According to a first aspect of the present disclosure, an intrathoracic pressure calculation device comprises a pulse wave acquisition unit to acquire a pulse wave signal obtained by measuring a pulse wave of a subject along a time axis. The intrathoracic pressure calculation device further comprises an intrathoracic pressure calculation unit to calculate an intrathoracic pressure of the subject based on the pulse wave signal acquired with the pulse wave acquisition unit. The intrathoracic pressure calculation device further comprises an intraoral pressure acquisition unit to acquire an intraoral pressure signal that indicates a magnitude of an intraoral pressure of the subject when the subject breathes with a different depth along the time axis, the intraoral pressure signal being associated with the pulse wave signal acquired with the pulse wave acquisition unit along the time axis. The intrathoracic pressure calculation device further comprises a coefficient calculation unit to calculate, as a calibration coefficient, a ratio of a variation in an amount of change in an amplitude of the pulse wave signal from a preset second reference value to a variation in an amount of change in the intraoral pressure represented by the intraoral pressure signal from a preset first reference value, based on the pulse wave signal acquired with the pulse wave acquisition unit and the intraoral pressure signal acquired with the intraoral pressure acquisition unit. The intrathoracic pressure calculation unit is to multiply an estimated intrathoracic pressure, which is a relative value of the intrathoracic pressure estimated based on the pulse wave signal acquired with the pulse wave acquisition unit, by the calibration coefficient, which is calculated with the coefficient calculation unit, to calculate an absolute value of the intrathoracic pressure of the subject.

According to another aspect of the present disclosure, an intrathoracic pressure calculation method comprises acquiring, in a pulse wave acquisition step, a pulse wave signal obtained by measuring a pulse wave of a subject along a time axis. The intrathoracic pressure calculation method further comprises calculating, in an intrathoracic pressure calculation step, an intrathoracic pressure of the subject based on the pulse wave signal acquired with the pulse wave acquisition step. The intrathoracic pressure calculation method further comprises acquiring, in an intraoral pressure acquisition step, an intraoral pressure signal that indicates a magnitude of an intraoral pressure of the subject when the subject breathes with a different depth along the time axis, the intraoral pressure signal being associated with the pulse wave signal acquired with the pulse wave acquisition step along the time axis. The intrathoracic pressure calculation method further comprises calculating, in a coefficient calculation step, as a calibration coefficient, a ratio of a variation in the amount of change in an amplitude of the pulse wave signal from a preset second reference value to a variation in the amount of change in the intraoral pressure represented by the intraoral pressure signal from a preset first reference value, based on the pulse wave signal acquired in the pulse wave acquisition step and the intraoral pressure signal acquired in the intraoral pressure acquisition step. The intrathoracic pressure calculation step includes multiplying an estimated intrathoracic pressure, which is a relative value of the intrathoracic pressure estimated based on the pulse wave signal acquired with the pulse wave acquisition step, by the calibration coefficient, which is calculated in the coefficient calculation step, to calculate an absolute value of the intrathoracic pressure of the subject.

### BRIEF DESCRIPTION OF THE DRAWINGS

The aforementioned object, other objects, characteristics, and advantages of the present disclosure become more apparent from a description that will be given with reference to the accompanying drawings. In the drawings,

FIG. 1 is a block diagram illustrating a schematic configuration of an intrathoracic pressure calculation system,

FIG. 2 is an illustrative view illustrating a schematic configuration of a breathing function inspection device,

FIG. 3 is a flowchart illustrating a processing procedure of a support process,

FIG. 4A is a diagram illustrating one example of an ideal breathing mode, and FIG. 4B is a diagram illustrating another example of the ideal breathing mode,

FIG. 5 is an illustrative diagram illustrating a processing outline of the support process,

FIG. 6 is a flowchart illustrating a processing procedure of a coefficient calculation process,

FIG. 7A is an illustrative diagram illustrating a transition of intraoral pressure due to breathing, and FIG. 7B is an illustrative view illustrating a transition of an estimated intrathoracic pressure due to breathing,

FIG. 8 is an illustrative view illustrating a technique of calculating a calibration coefficient,

FIG. 9 is a flowchart illustrating a processing procedure of an intrathoracic pressure calculation process, and

FIG. 10 is a graph of experimental results illustrating a basic concept of a method of calculating the calibration coefficient.

### DESCRIPTION OF EMBODIMENTS

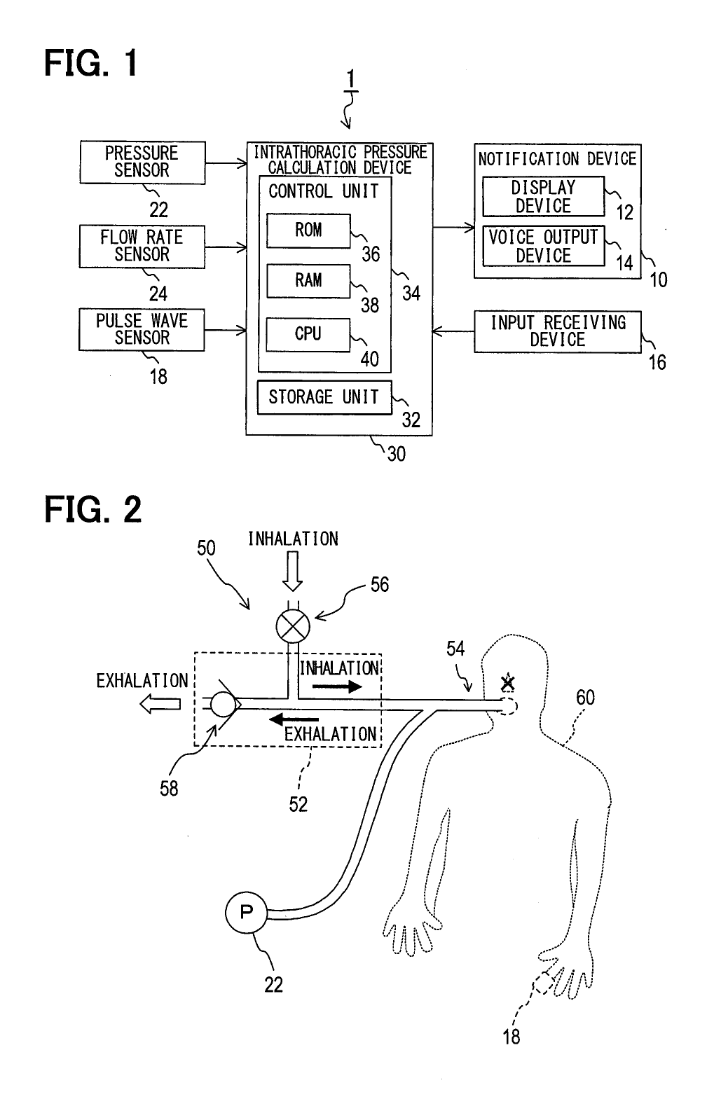

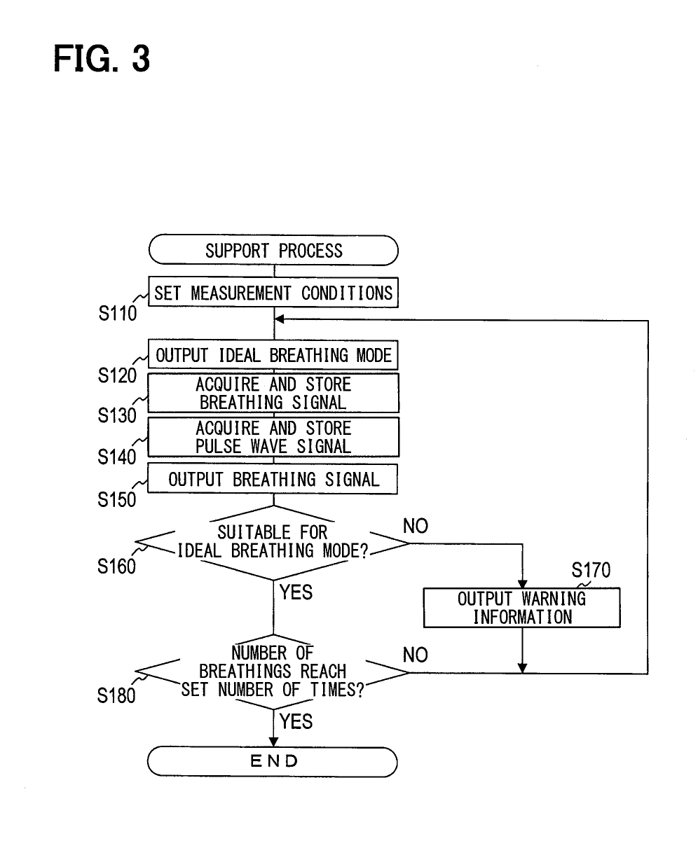

Hereinafter, the embodiments of the present disclosure will be described with reference to the drawings. An intrathoracic pressure calculation system 1 illustrated in FIG. 1 is a system for converting an estimated intrathoracic pressure estimated based on a pulse wave signal representing a pulse wave of a subject 60 (refer to FIG. 2) to an absolute value of an intrathoracic pressure of the subject 60. The intrathoracic pressure is a pressure in a thoracic space of the subject 60. In addition, the estimated intrathoracic pressure represents a transition of a pressure based on a relative change in an amplitude of the pulse wave signal, and the estimated intrathoracic pressure is a relative value of the intrathoracic pressure.

Subsequently, a control unit 34 outputs a notification signal indicating an ideal breathing mode to a notification device 10 (S120). The ideal breathing mode referred to in the present disclosure is an ideal breathing mode for measuring an intraoral pressure, a ventilation amount, and the pulse wave signal which are necessary for executing a coefficient calculation process. The ideal breathing referred to in the present disclosure is directed to a resting breathing, but may be other breathing. In other words, the ideal breathing mode is one of the modes of the resting breathing implemented by the subject 60, and is predefined as a breathing mode in which breathing different in depth is implemented multiple times.

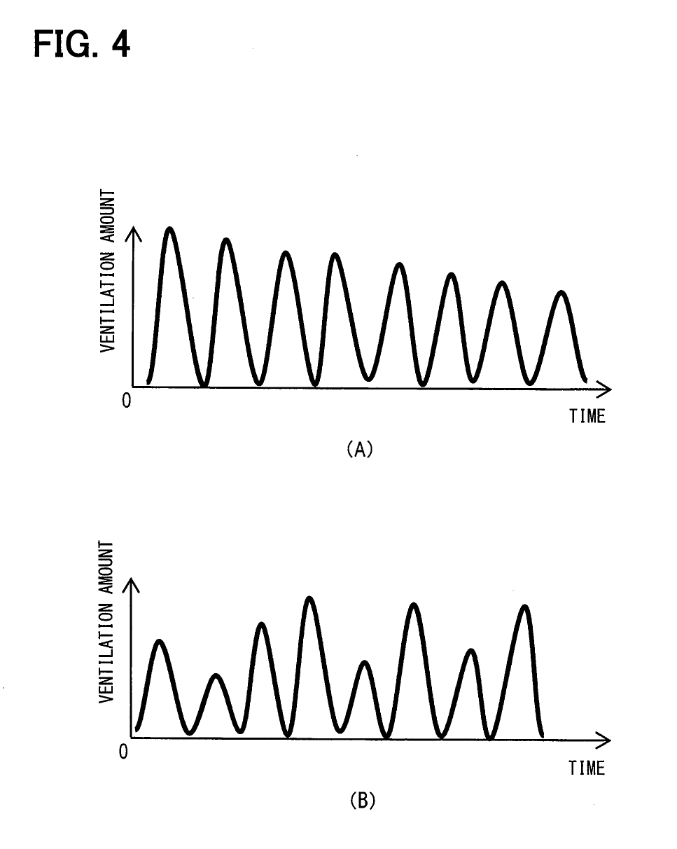

As an example of the ideal breathing mode in the present embodiment, it is conceivable that the ventilation amount when performing multiple breathing is changed while a magnitude of a resistance set by a resistance setting unit 56 of a breathing function inspection device 50 is kept constant. In this case, as shown in FIG. 4A, the amount of ventilation may be defined to decrease as time progresses, or as shown in FIG. 4B, the different ventilation amount may be defined along a time axis at random. In those cases, it is preferable that at least two or more levels of flow rates are set for the ventilation amount.

As another example of the ideal breathing mode in the present embodiment, it is conceivable that the magnitude of the resistance set by the resistance setting unit 56 in the breathing function inspection device 50 is changed each time the subject 60 performs resting breathing at a required number of times, while the ventilation amount when the subject 60 performs breathing is kept constant. In that case, it is preferable that the magnitude of the resistance set by the resistance setting unit 56 has at least two or more levels.

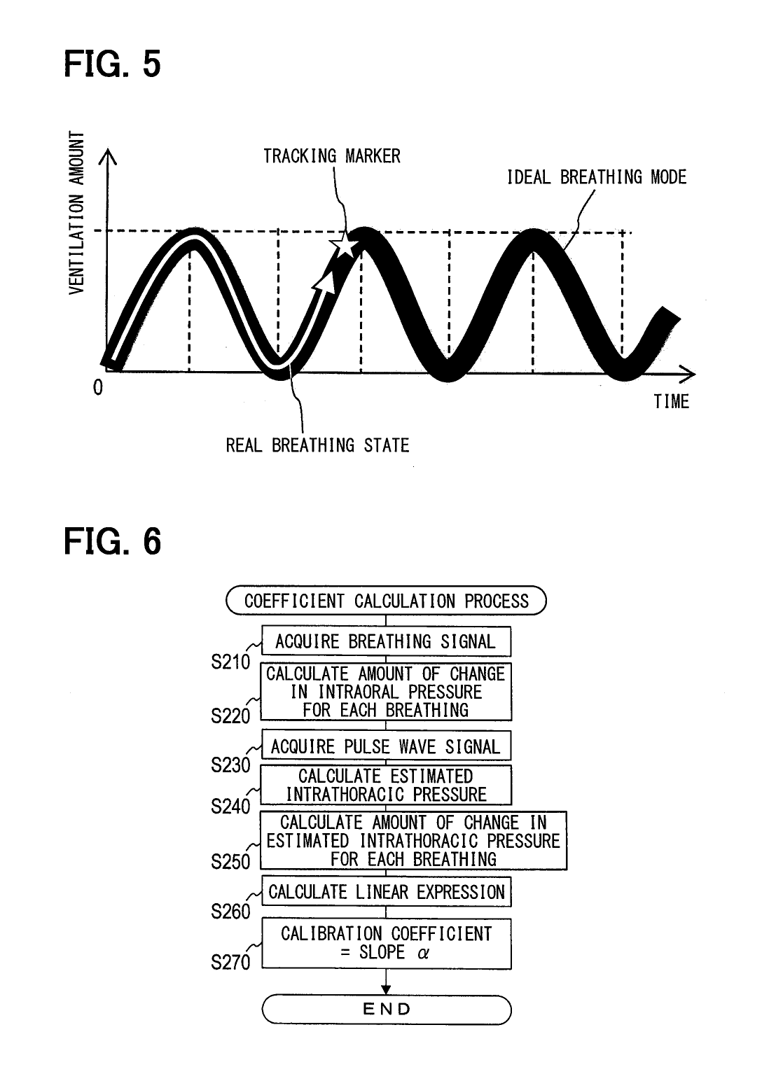

The notification device 10 that has acquired the notification signal notifies the ideal breathing mode indicated by the acquired notification signal. Specifically, a display device 12 displays, as the ideal breathing mode, a correspondence relationship between the amount of breathing (that is, ventilation amount) to be inhaled and exhaled by the subject 60 and time, as shown in FIG. 5. The ideal breathing mode displayed by the display device 12 may indicate a tracking marker indicating a standard when the subject 60 breathes along a time axis.

In addition, the notification device 10 that has acquired the notification signal may output the ideal breathing mode represented by the acquired notification signal as a voice. The subject 60 breathes so as to approach the ideal breathing mode.

In a support process, the control unit 34 acquires a breathing signal and stores the breathing signal in a storage unit 32 (S130). The breathing signal referred to in the present disclosure represents a state of breathing actually performed by the subject 60. The breathing signal is indicative of results measured by a pressure sensor 22 and a flow rate sensor 24. In other words, the breathing signal includes an intraoral pressure signal and a transition of a ventilation amount.

In the breathing signal, the intraoral pressure signal represents a result measured by the pressure sensor 22, and serves as a signal representing the transition of the intraoral pressure of the subject 60 with the repetitive execution of S130 in the support process.

The control unit 34 acquires the pulse wave signal and stores the pulse wave signal in the storage unit 32 (S140). The pulse wave signal represents a result measured by a pulse wave sensor 18. The pulse wave signal is a signal representing a transition of the pulse wave when the subject 60 is actually breathing with the repetitive execution of S140 in the support process. The pulse wave signal acquired in S140 of the present embodiment is associated with at least the intraoral pressure signal acquired in S130 along a time axis.

Subsequently, the control unit 34 outputs the breathing signal acquired in S130 to the notification device 10 (S150). The notification device 10 that has acquired the breathing signal notifies the acquired breathing signal. For example, as shown in FIG. 5, the display device 12 superimposes a real breathing state based on the transition of the ventilation amount of the breathing signal on the ideal breathing mode for display. The real breathing state referred to in the present disclosure represents a state of breathing represented by the ventilation amount and the intraoral pressure, which is a state of breathing actually performed by the subject 60.

Further, in the support process, the control unit 34 determines whether the real breathing state falls within an allowable range as the ideal breathing mode, or not (S160). As a result of the determination in S160, when the real breathing mode falls within the allowable range as the ideal breathing mode (yes in S160), the control unit 34 shifts the support process to S180 to be described in detail later.

On the other hand, as a result of the determination in S160, when the real breathing mode does not fall within the allowable range as the ideal breathing mode (no in S160), the control unit 34 shifts the support process to S170. In S170, the control unit 34 outputs warning information indicating that the real breathing mode does not fall within the allowable range as the ideal breathing mode to the notification device 10.

The notification device 10 that has acquired the warning information notifies that the real breathing mode does not fall within the allowable range as the ideal breathing mode. As an example of the notification content, an advice for bringing the real breathing mode closer to the ideal breathing mode can be considered.

Thereafter, the control unit 34 returns the support process to S120 and executes the subsequent steps in the support process. Incidentally, in S180 shifted when the real breathing mode falls within the allowable range as the ideal breathing mode as a result of the determination in S160, the control unit 34 determines whether the number of breathings performed by the subject 60 has reached a set number of times set in S110, or not. As a result of the determination in S180, when the number of breaths has not reached the set number of times (no in S180), the control unit 34 returns the support process to S120 and executes the subsequent steps in the support process.

On the other hand, as a result of the determination in S180, when the number of breaths has reached the set number value (yes in S180), the control unit 34 completes the support process. In other words, in the support process, the control unit 34 notifies the ideal breathing mode. During a period in which the subject 60 is breathing, the control unit 34 senses the intraoral pressure, the ventilation amount, and the pulse wave. Further, in the support process, the control unit 34 stores the results of sensing in association with each other along the time axis.

Next, the coefficient calculation process to be executed by the control unit 34 of an intrathoracic pressure calculation device 30 will be described. The coefficient calculation process is started when a calculation start command is inputted through an input receiving device 16. The calculation start command is a command to start the coefficient calculation process. When the coefficient calculation process is started, as shown in FIG. 6, the control unit 34 acquires the breathing signal stored in S130 of the support process (S210). Subsequently, the control unit 34 calculates the amount of change in the intraoral pressure for each breath based on the intraoral pressure signal of the breathing signal acquired in S210 (S220).

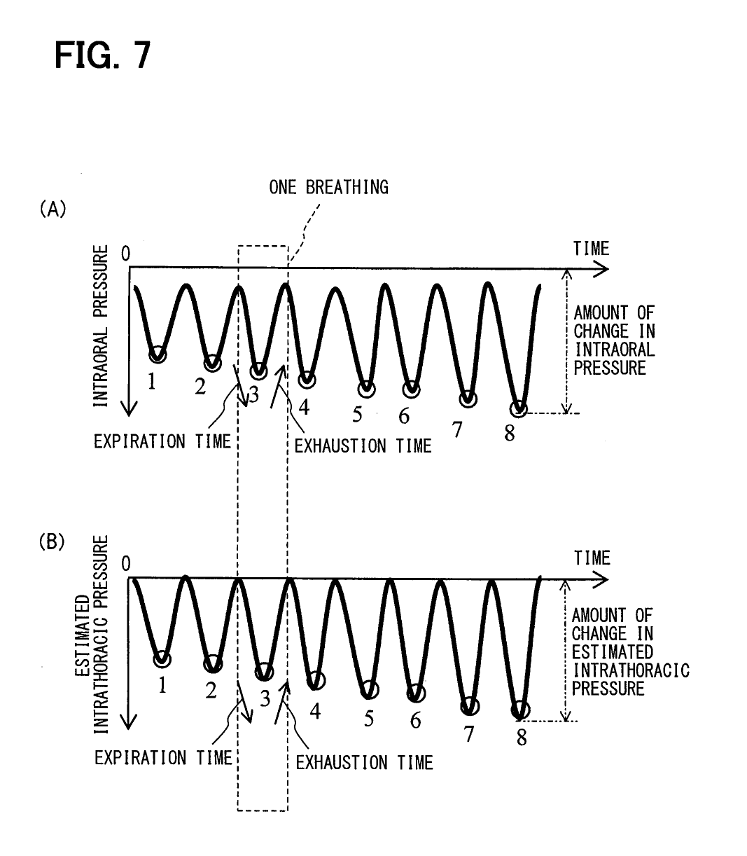

Specifically, in S220 of the present embodiment, as shown in FIG. 7A, in the transition of the intraoral pressure represented by the intraoral pressure signal, the control unit 34 calculates a difference between a peak of the intraoral pressure signal in each breathing and a first reference value as the amount of change in the intraoral pressure in each breathing. The first reference value referred to in the present disclosure is a preset value of the intraoral pressure. As an example of the first reference value, a value of pressure equal to the atmospheric pressure (that is, “0” shown in FIG. 7A) or an intraoral pressure at an end of expiration can be considered.

Subsequently, in the coefficient calculation process, the control unit 34 acquires the pulse wave signal stored in S140 of the support process (S230). Subsequently, the control unit 34 calculates an estimated intrathoracic pressure based on the pulse wave signal acquired in S230 (S240).

As a technique of estimating the estimated intrathoracic pressure in S240, since a well-known technique may be used, a detailed description of the estimation technique will be omitted in the present disclosure. However, as an example of a technique for estimating the estimated intrathoracic pressure, a technique disclosed in Japanese Unexamined Patent Application Publication No. 2002-355227 is conceivable. In other words, in the estimation of the estimated intrathoracic pressure, first, the control unit 34 creates a first envelope obtained by connecting peaks of an amplitude in one pulse wave represented by the pulse wave signal, and a second envelope obtained by connecting peaks of the first envelope. The control unit 34 may calculate a difference between the first envelope and the second envelope as the estimated intrathoracic pressure.

Furthermore, in the coefficient calculation process, the control unit 34 calculates the amount of change in the estimated intrathoracic pressure every breath based on the estimated intrathoracic pressure calculated in S240 (S250). Specifically, in S250 of the present embodiment, as shown in FIG. 7B, the control unit 34 calculates a difference between the peak of the estimated intrathoracic pressure at each breathing and a second reference value as the amount of change in the estimated intrathoracic pressure in each breathing. The second reference value referred to in the present disclosure is a value of the estimated intrathoracic pressure set in advance. As an example of the second reference value, a value of pressure equal to the atmospheric pressure (that is, “0” shown in FIG. 7B) or an intrathoracic pressure at an end of expiration can be considered.

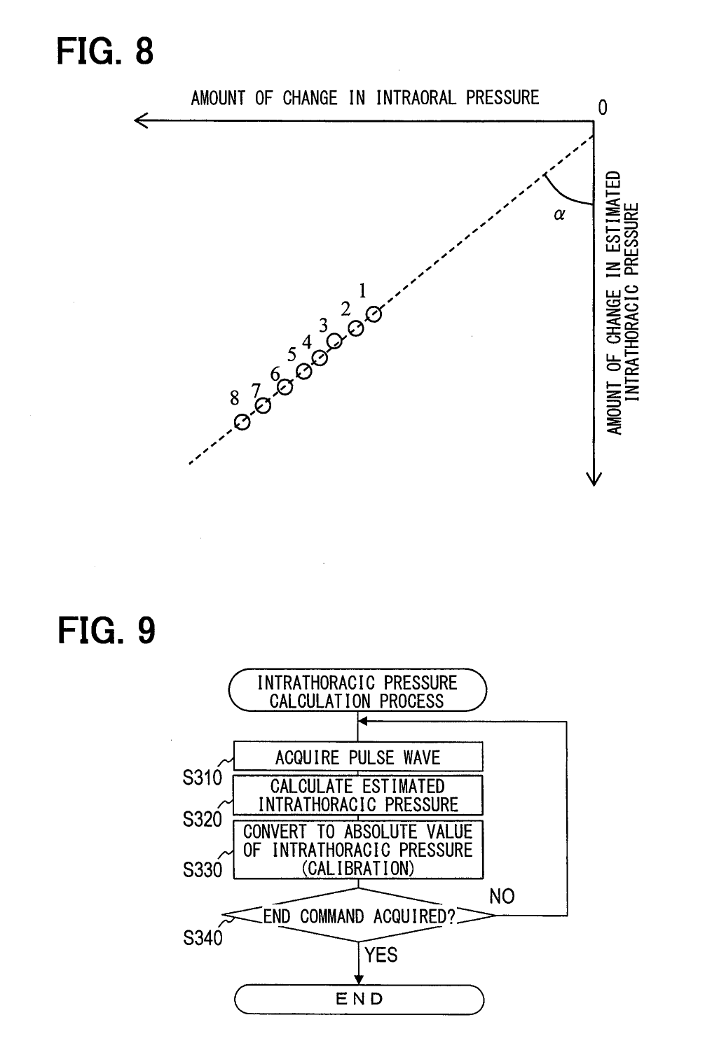

Further, the control unit 34 calculates a correspondence relationship between the amount of change in the intraoral pressure and the amount of change in the estimated intrathoracic pressure by a linear expression (S260). In the calculation of the linear expression in S260, as shown in FIG. 8, first, the control unit 34 develops (plots) the amount of change in the intraoral pressure calculated in S220 and the amount of change in the estimated intrathoracic pressure calculated in S250 on two-dimensional plane for each same breathing. Then, the control unit 34 executes a well-known linear regression analysis for obtaining the linear expression on the developed amount of change in the intraoral pressure and the developed amount of change in the estimated intrathoracic pressure. A representative example of the linear regression analysis is the least squares method.

As a result, the linear expression expressing a correspondence relationship between the amount of change in the intraoral pressure and the amount of change in the estimated intrathoracic pressure is calculated. Subsequently, the control unit 34 sets a slope a of the linear expression calculated in S260 as a calibration coefficient (S270). In other words, in S270 of the coefficient calculation process, the control unit 34 sets, as the calibration coefficient, the ratio of the variation in the amount of change in the estimated intrathoracic pressure to the variation in the amount of change in the intraoral pressure. In other words, the ratio of the variation in the amount of change in the estimated intrathoracic pressure to the variation in the amount of change in the intraoral pressure is the slope a between the amount of change in the intraoral pressure and the amount of change in the estimated intrathoracic pressure.

Thereafter, the coefficient calculation process is completed.

Next, an intrathoracic pressure calculation process to be executed by the control unit 34 of the intrathoracic pressure calculation device 30 will be described. The intrathoracic pressure calculation process is started when an internal pressure calculation start command is inputted through the input receiving device 16. The internal pressure calculation start command is a command to start the intrathoracic pressure calculation process. When the intrathoracic pressure calculation process is started, as shown in FIG. 9, first, the control unit 34 acquires the pulse wave (pulse wave signal) detected by the pulse wave sensor 18 (S310).

Subsequently, the control unit 34 calculates the estimated intrathoracic pressure based on the pulse wave acquired in S310 (S320). As a technique of estimating the estimated intrathoracic pressure in S320, as in S240 of the coefficient calculation process, since a well-known technique may be used, a detailed description of the estimation technique will be omitted in the present disclosure. However, as an example of a technique for estimating the estimated intrathoracic pressure, a technique disclosed in Japanese Unexamined Patent Application Publication No. 2002-355227 is conceivable. In other words, in the estimation of the estimated intrathoracic pressure, first, the control unit 34 creates a first envelope obtained by connecting peaks of one pulse wave represented by the pulse wave signal, and a second envelope obtained by connecting peaks of the first envelope. The control unit 34 may calculate a difference between the first envelope and the second envelope as the estimated intrathoracic pressure.

Then, the control unit 34 calculates an absolute value of the estimated intrathoracic pressure of the subject 60 (S330). Specifically, in S330 of the present embodiment, the control unit 34 calculates the absolute value of the intrathoracic pressure of the subject 60 by multiplying the estimated intrathoracic pressure calculated in S320 by the calibration coefficient set in S270 of the coefficient calculation process.

Further, the control unit 34 determines whether to accept an input of an end command for completing the intrathoracic pressure calculation process, or not (S340). As a result of the determination, when the termination command has not been accepted (no in S340), the control unit 34 returns the intrathoracic pressure calculation process to S310, and calculates the absolute value of the intrathoracic pressure of the subject 60 based on the newly acquired pulse wave.

On the other hand, as a result of the determination in S340, when the end command has been accepted (yes in S340), the control unit 34 completes the intrathoracic pressure calculation process.

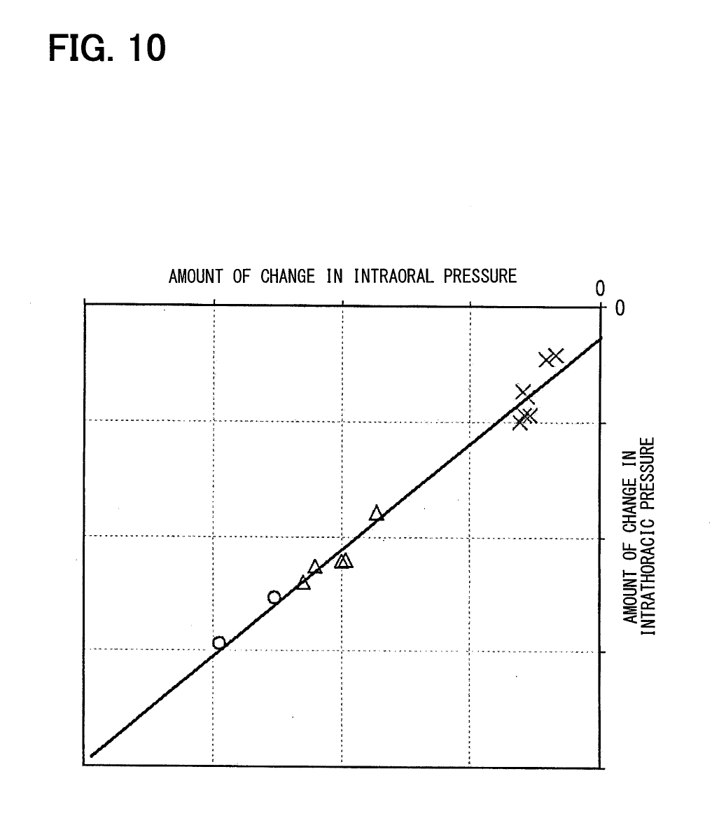

As a result of intensive research by the inventors, as shown in FIG. 10, it has been found that the amount of change in the intraoral pressure of the subject 60 in the resting breathing from the first reference value is equal to the amount of change in the intrathoracic pressure from the second reference value regardless of a magnitude of a resistance between the oral cavity and the thoracic cavity.

Based on the above finding, in the coefficient calculation process, the control unit 34 derives the variation in the amount of change in the amplitude of the pulse wave signal from the second reference value to the variation in the amount of change in the intraoral pressure from the first reference value as the calibration coefficient.

In other words, the calibration coefficient multiplied by the estimated intrathoracic pressure in the intrathoracic pressure calculation process is a correction coefficient for converting the relative value of the intrathoracic pressure to the absolute value of the intrathoracic pressure irrespective of the magnitude of the resistance between the oral cavity and the thoracic cavity.

Therefore, according to the intrathoracic pressure calculation processing, the calculation accuracy of the intrathoracic pressure can be improved. In particular, in the coefficient calculation process, the control unit 34 derives the slope a between the amount of change in the intraoral pressure from the first reference value and the amount of change in the estimated intrathoracic pressure from the second reference value in each of two or more breathings different in depth as the calibration coefficient.

Therefore, according to the coefficient calculation process, the calibration coefficient can be reliably calculated by a simple method. Further, in the support process, the ideal breathing mode is notified. For that reason, the subject 60 can recognize the ideal breathing mode and breathe in a mode close to the ideal breathing mode.

In the support process, the pulse wave signal and the intraoral pressure signal measured in a period during which the ideal breathing mode is notified, that is, when the subject 60 is breathing in the ideal breathing mode are acquired. Because the calibration coefficient is obtained in the coefficient calculation process based on the pulse wave signal and the intraoral pressure signal thus acquired, the calculation accuracy of the calibration coefficient can be more increased.

As a result, according to the intrathoracic pressure calculation process, the calculation accuracy of the intrathoracic pressure can be more increased. In the support process, it is conceivable that the magnitude of the resistance set by the resistance setting unit 56 in the breathing function inspection device 50 is changed each time the subject 60 performs resting breathing at a required number of times, while the ventilation amount when the subject 60 breathes is kept constant, to thereby attain the ideal breathing mode. In that case, because the ventilation amount of the breathing performed by the subject 60 may be kept constant, the ideal breathing mode can be easily attained.

On the other hand, in the support process, it is conceivable that the ventilation amount when performing multiple breathing is changed while a magnitude of a resistance set by the resistance setting unit 56 of the breathing function inspection device 50 is kept constant, to thereby attain the ideal breathing mode. In that case, burden of changing the magnitude of the resistance set by the resistance setting unit 56 can be reduced.

### Other Embodiments

The embodiments of this disclosure have been described above. However, the present disclosure is not limited to the embodiments described above, and various modifications can be implemented without departing from the spirit of the present disclosure.

For example, the breathing function inspection device 50 in the above embodiment is provided with the flow rate sensor 24. However, the flow sensor 24 may not be provided in the breathing function inspection device 50.

Modes in which a part of the configurations of the above embodiments may be omitted are also encompassed by the embodiments of the present disclosure. Modes configured by appropriate combinations of the above embodiments with the modification are also encompassed by the embodiments of the present disclosure. Moreover, all modes considerable without departing from the essence of disclosure identified by wording described in the claims are encompassed by the embodiments of the present disclosure.

In addition to the intrathoracic pressure calculation device 30 described above, the present disclosure can be attained by various configurations such as the intrathoracic pressure calculation system 1 having the intrathoracic pressure calculation device 30 as a component, a program for causing a computer to function as the intrathoracic pressure calculation device 30, a medium storing the program, and a method of calculating the intrathoracic pressure.

As described above, the present disclosure relates to the intrathoracic pressure calculation device including a pulse wave acquisition unit, an intrathoracic pressure calculation unit, an intraoral pressure acquisition unit, and a coefficient calculation unit. The pulse wave acquisition unit acquires the pulse wave signal obtained by measuring the pulse wave of the subject along the time axis. The intrathoracic pressure calculation unit calculates the intrathoracic pressure of the subject based on the pulse wave signal acquired with the pulse wave acquisition unit. Furthermore, the intraoral pressure acquisition unit acquires the intraoral pressure signal representing the magnitude of the intraoral pressure of the subject when the subject breathes differently in depth along the time axis. In this example, the intraoral pressure signal acquired with the intraoral pressure acquisition unit is associated with the pulse wave signal acquired with the pulse wave acquisition unit along the time axis. The coefficient calculation unit calculates the calibration coefficient based on the intraoral pressure signal acquired with the intraoral pressure acquisition unit and the pulse wave signal acquired with the pulse wave acquisition unit. The calibration coefficient is the ratio of the variation in the amount of change in the amplitude of the pulse wave signal from the second reference value set in advance to the variation in the amount of change in the intraoral pressure represented by the intraoral pressure signal from the first reference value set in advance. The intrathoracic pressure calculation unit multiplies the estimated intrathoracic pressure which is the relative value of the intrathoracic pressure estimated based on the pulse wave signal acquired with the pulse wave acquisition unit by the calibration coefficient calculated by the coefficient calculation unit, to thereby estimate the absolute value of the intrathoracic pressure of the subject.

As a result of intensive research by the inventors, it has been found that the amount of change in the intraoral pressure of the subject from the first reference value set in advance is equal to the amount of change in the intrathoracic pressure from the reference value set in advance regardless of the magnitude of the resistance between the oral cavity and the thoracic cavity when the breathing falls within the resting breathing. Based on the above finding, in the intrathoracic pressure calculation device, the variation in the amount of change in the amplitude of the pulse wave signal from the second reference value to the variation in the amount of change in the intraoral pressure from the first reference value is derived as the calibration coefficient. In other words, the calibration coefficient multiplied by the estimated intrathoracic pressure in the intrathoracic pressure calculation device is a correction coefficient for converting the relative value of the intrathoracic pressure to the absolute value of the intrathoracic pressure irrespective of the magnitude of the resistance between the oral cavity and the thoracic cavity. Then, in the intrathoracic pressure calculation device, the relative value of the estimated intrathoracic pressure estimated based on the pulse wave signal is converted into the absolute value of the intrathoracic pressure of the subject. Therefore, according to the intrathoracic pressure calculation device, the calculation accuracy of the intrathoracic pressure can be improved.

Incidentally, the present disclosure may be made as a calculation method for calculating the intrathoracic pressure.

According to the intrathoracic pressure calculation method described above, the same advantages as those of the intrathoracic pressure calculation device can be obtained.

The present disclosure has been described based on examples, but it is understood that the present disclosure is not limited to the examples or structures. The present disclosure includes various modification examples and modifications within the same range. In addition, it should be understood that various combinations or aspects, or other combinations or aspects, in which only one element, one or more elements, or one or less elements is included to the various combinations or aspects, are included in the scope or the technical idea of the present disclosure.

1. An intrathoracic pressure calculation device comprising:
a pulse wave acquisition unit to acquire a pulse wave signal obtained by measuring a pulse wave of a subject along a time axis;
an intrathoracic pressure calculation unit to calculate an intrathoracic pressure of the subject based on the pulse wave signal acquired with the pulse wave acquisition unit;
an intraoral pressure acquisition unit to acquire an intraoral pressure signal that indicates a magnitude of an intraoral pressure of the subject when the subject breathes with a different depth along the time axis, the intraoral pressure signal being associated with the pulse wave signal acquired with the pulse wave acquisition unit along the time axis; and
a coefficient calculation unit to calculate, as a calibration coefficient, a ratio of a variation in an amount of change in an amplitude of the pulse wave signal from a preset second reference value to a variation in an amount of change in the intraoral pressure represented by the intraoral pressure signal from a preset first reference value, based on the pulse wave signal acquired with the pulse wave acquisition unit and the intraoral pressure signal acquired with the intraoral pressure acquisition unit, wherein
the intrathoracic pressure calculation unit is to multiply an estimated intrathoracic pressure, which is a relative value of the intrathoracic pressure estimated based on the pulse wave signal acquired with the pulse wave acquisition unit, by the calibration coefficient, which is calculated with the coefficient calculation unit, to calculate an absolute value of the intrathoracic pressure of the subject.

2. The intrathoracic pressure calculation device according to claim 1, wherein
the coefficient calculation unit includes:
an intraoral pressure change amount calculation unit to calculate the amount of change in the intraoral pressure from the first reference value in each breathing based on the intraoral pressure signal acquired with the intraoral pressure acquisition unit; and
a breathing change amount calculation unit to calculate the amount of change in the amount of breathing from the second reference value in each breathing, based on the pulse wave signal acquired with the pulse wave acquisition unit, wherein
the coefficient calculation unit is to derive, as the calibration coefficient, a slope defined between the amount of change in the intraoral pressure from the first reference value in each breathing and the amount of change in the amount of breathing from the second reference value in each breathing.

3. The intrathoracic pressure calculation device according to claim 1, further comprising:
a notification unit to notify an ideal breathing mode to be implemented by the subject, the ideal breathing mode being predefined as a breathing mode having different depths.

4. The intrathoracic pressure calculation device according to claim 3, wherein
the pulse wave acquisition unit is to acquire the pulse wave signal measured in a period during which the notification unit is notifying the ideal breathing mode, and
the intraoral pressure acquisition unit is to acquire the intraoral pressure signal measured in the period during which the notification unit is notifying the ideal breathing mode.

5. The intrathoracic pressure calculation method according to claim 3, wherein
the ideal breathing mode is to attain the breathing different in the depth by allowing the subject to breath with a ventilation amount which is a flow rate of air when the subject performs breathing, and which is a ventilation amount of a predefined flow rate, through a resistance different in magnitude.

6. The intrathoracic pressure calculation device according to claim 5, wherein
the magnitude of the resistance has at least two levels.

7. The intrathoracic pressure calculation device according to claim 3, wherein
the ideal breathing mode is to attain the breathing different in the depth with change in a ventilation amount, which is a flow rate of air when the subject performs breathing.

8. The intrathoracic pressure calculation device according to claim 7, wherein
the ventilation amount is a flow rate having at least two levels.

9. An intrathoracic pressure calculation method comprising:
acquiring, in a pulse wave acquisition step, a pulse wave signal obtained by measuring a pulse wave of a subject along a time axis;
calculating, in an intrathoracic pressure calculation step, an intrathoracic pressure of the subject based on the pulse wave signal acquired with the pulse wave acquisition step;
acquiring, in an intraoral pressure acquisition step, an intraoral pressure signal that indicates a magnitude of an intraoral pressure of the subject when the subject breathes with a different depth along the time axis, the intraoral pressure signal being associated with the pulse wave signal acquired with the pulse wave acquisition step along the time axis; and
calculating, in a coefficient calculation step, as a calibration coefficient, a ratio of a variation in the amount of change in an amplitude of the pulse wave signal from a preset second reference value to a variation in the amount of change in the intraoral pressure represented by the intraoral pressure signal from a preset first reference value, based on the pulse wave signal acquired in the pulse wave acquisition step and the intraoral pressure signal acquired in the intraoral pressure acquisition step, wherein
the intrathoracic pressure calculation step includes multiplying an estimated intrathoracic pressure, which is a relative value of the intrathoracic pressure estimated based on the pulse wave signal acquired with the pulse wave acquisition step, by the calibration coefficient, which is calculated in the coefficient calculation step, to calculate an absolute value of the intrathoracic pressure of the subject.
